(19) United States
(12) Patent Application Publication
(10) Pub. No.: US 2015/0231325 A1
(43) Pub. Date: Aug. 20, 2015

(54) POWER INJECTOR WITH MOVABLE JOINT-INTEGRATED SIGNAL TRANSMISSION CONNECTOR

(71) Applicant: Mallinckrodt LLC, Hazelwood, MO (US)

(72) Inventors: Charles S. Neer, Cincinnati, OH (US); Louis Francis Wourms, Beavercreek, OH (US)

(73) Assignee: MALLINCKRODT LLC, Hazelwood, MO (US)

(21) Appl. No.: 14/677,584

(22) Filed: Apr. 2, 2015

Related U.S. Application Data
(63) Continuation of application No. 12/179,129, filed on Jul. 24, 2008.
(60) Provisional application No. 60/970,599, filed on Sep. 7, 2007.

(51) Int. Cl. A61M 5/00; A61M 5/14; A61M 5/178; A61M 5/145

(52) U.S. Cl. CPC A61M 5/007; A61M 5/14546; A61M 5/1408; A61M 5/178; A61M 2209/084

(57) ABSTRACT

A power injector that incorporates at least one signal transmission connector in a movable joint (e.g., between a powerhead and a support or stand; between at least one adjacent pair of support sections of a support for the powerhead) is disclosed. Such a signal transmission connector may be in the form of a slip ring module, but in any case is part of the signal transmission link to the powerhead. At least that portion of a signal transmission conduit that is adjacent to the powerhead may be disposed within the interior of the corresponding portion of the support or stand, thereby reducing the potential that an individual will grab onto the signal transmission conduit when attempting to move at least part of the power injector, to change the position of the powerhead, or both.

### RELATED APPLICATIONS

This application is a continuation of U.S. patent application Ser. No. 12/179,129, filed on Jul. 24, 2008, which claims priority to and is a non-provisional application of U.S. Provisional Application Ser. No. 60/970,599 filed on Sep. 7, 2007 and entitled POWER INJECTOR WITH MOVABLE JOINT-INTEGRATED SIGNAL TRANSMISSION CONNECTOR. The entire disclosure of each patent application that is set forth in this Related Applications section is hereby incorporated by reference.

### FIELD OF THE INVENTION

The present invention generally relates to the field of power injectors and, more particularly, to the manner of providing signals to a powerhead of a power injector.

### BACKGROUND

Various medical procedures require that one or more fluids be injected into the patient. Medical imaging procedures oftentimes involve the injection of contrast media into the patient, possibly along with saline or other fluids. Power injectors may be used for these types of applications.

A power injector generally includes what is commonly referred to as a powerhead. One or more syringes may be mounted to the powerhead in various manners (e.g., detachably). Each syringe typically includes what may be characterized as a syringe plunger, piston, or the like. Each such syringe plunger is appropriately interconnected with an appropriate syringe driver that is incorporated into the powerhead, such that operation of the syringe driver axially advances the associated syringe plunger. One typical syringe driver is in the form of a ram mounted on a threaded lead screw. Rotation of the lead screw in one rotational direction advances the associated ram in one axial direction, while rotation of the lead screw in the opposite rotational direction advances the associated ram in the opposite axial direction.

An electrical motor or the like may be the drive source for the various syringe drivers of the powerhead. Other electrical components may be incorporated into the powerhead. As such, there is commonly a need to provide electrical power to the powerhead. A cable with multiple wires may be attached to the powerhead to provide the desired electrical power. One known configuration attaches a cable to both the powerhead and a support of the power injector. Since the powerhead may need to be moved through at least a certain range of motion for one or more purposes (e.g., loading one or more fluids into a corresponding syringe attached to the powerhead; injecting one or more fluids from a corresponding syringe attached to the powerhead), the cable may include a “loop” between its attachment locations at the support and powerhead. Although this loop may facilitate movement of the powerhead, movement of the powerhead relative to its corresponding support will still typically stress the cable to at least some degree and may eventually impair one or more electrical connections. Having the cable be exteriorly disposed also increases the likelihood that the cable will be grabbed to pull on the power injector to move the power injector from one location to another.

### SUMMARY

A first aspect of the present invention is embodied by a power injector having a support, a signal transmission conduit, a powerhead, and a movable joint. The movable joint is disposed between the support and the powerhead to allow the powerhead to move relative to the support (e.g., at least generally about a first axis). The movable joint includes a signal transmission connector that operatively interconnects the signal transmission conduit with the powerhead (e.g., this signal transmission connector may be characterized as being part of a signal transmission link extending to the powerhead). This signal transmission connector includes first and second components that move relative to each other (e.g., at least generally about the first axis) responsive to a movement of the powerhead relative to the support.

A second aspect of the present invention is embodied by a power injector having a support, a powerhead, a signal transmission link extending to the powerhead, and at least one movable joint that is associated with at least one of the support and the powerhead. At least one movable joint includes or incorporates a signal transmission connector that is part of the signal transmission link to the powerhead. This signal transmission connector includes first and second components that move relative to each other (e.g., at least generally about a first axis) responsive to a movement of at least one of the support and the powerhead.

A third aspect of the present invention is embodied by a power injector having a support, a signal transmission conduit, a powerhead, and a signal transmission connector. At least that portion of the signal transmission conduit that is adjacent to the powerhead is disposed within an interior of the support. The signal transmission connector is operatively interconnected with the signal transmission conduit, and includes first and second components that move relative to each other responsive to a movement of at least one of the support and the powerhead.

The remainder of this Summary pertains to each of the first, second, and third aspects, unless otherwise noted. Various refinements exist of the features noted in relation to each of the above-noted first, second, and third aspects of the present invention. Further features may also be incorporated in each of the above-noted first, second, and third aspects of the present invention as well. These refinements and additional features may exist individually or in any combination in relation to each of the first, second, and third aspects.

The power injector may be used for any appropriate application where the delivery of one or more fluids is desired, including without limitation any appropriate medical application (e.g., computed tomography or CT imaging; magnetic resonance imaging or MRI; SPECT imaging; PET imaging; X-ray imaging; angiographic imaging; optical imaging; ultrasound imaging). The power injector may be used in conjunction with any component or combination of components, such as an appropriate imaging system (e.g., a CT scanner). For instance, information could be conveyed between the power injector and one or more other components (e.g., scan delay information, injection start signal, injection rate). Any appropriate number of syringes may be integrated with the powerhead in any appropriate manner (e.g., detachably), and any appropriate fluid may be discharged from a multiple syringe configuration in any appropriate manner (e.g., sequentially, simultaneously).

The support may be of any appropriate size, shape, configuration, and/or type. The support may be either adjustable or non-adjustable. For instance, an adjustable support may allow the position of one portion of the support to be adjusted relative to the position of another portion of the support. In one embodiment, the support is mounted to an appropriate surface or structure, such as a wall, ceiling, or floor. In another embodiment, the support includes a portable base and a column that extends at least generally upwardly from the base. At least part of the signal transmission conduit may be disposed within the column. Any such base may include wheels, rollers, or any other appropriate structure to provide a transportability feature for the power injector.

At least that part of a signal transmission conduit that is adjacent to the powerhead may be disposed within the interior of the support. This reduces the potential for grasping the signal transmission conduit to move at least part of the power injector, to adjust the position/orientation of the powerhead, or both. The signal transmission conduit may be in the form of one or more cables that are operatively interconnected in any appropriate manner. Each such cable may include any appropriate number of individual wires to provide a desired number of signal transmission links with the powerhead or other adjacent structure. The signal path extending to the powerhead may be characterized as a signal transmission link, which in turn may include the noted signal transmission conduit, one or more signal transmission connectors, or both.

Any movable joint that incorporates a signal transmission connector may be of any appropriate size, shape, configuration, and/or type. In one embodiment, such a movable joint is in the form of a pivot. In another embodiment, the movable joint is of a configuration that constrains motion to at least substantially within a single dimension. In the case where such a movable joint is between the powerhead and the support, this would then allow the powerhead to move relative to an adjacent portion of the support. The powerhead may move through any appropriate range of motion, regardless of the configuration of the movable joint.

A movable joint that incorporates a signal transmission connection may also be disposed between at least one adjacent pair of support sections of the support to allow the same to move relative to each other (e.g., relative motion being constrained to at least substantially within a single dimension). For instance, this support could be mounted to an appropriate structure, such as a wall, ceiling, or floor. One such movable joint could be disposed between the powerhead and an adjacent portion of the support, while another such movable joint could be disposed between at least one pair of adjacent support sections of the support to allow the same to move relative to each other (e.g., relative motion being constrained to at least substantially within a single dimension).

The signal transmission connector may be of any appropriate size, shape, configuration, and/or type. The signal transmission connector may be characterized as a slip ring module, as a rotary connector, or both. In one embodiment, the signal transmission connector includes first and second components (e.g., housings) that are movable relative to each other. The first and second components may move relative to each other in any appropriate manner, including through a relative rotational or pivotal movement about an axis. This axis may coincide with an axis about which different portions of the power injector move relative to each other. For instance, the first component may be associated with a signal transmission conduit (e.g., the signal transmission conduit may be interconnected with the first component) and the second component may be associated with the powerhead (e.g., the second component may be interconnected with the powerhead, for instance, such that the second component may move along with the powerhead).

A fourth aspect of the present invention is embodied by a power injector having a support, a signal transmission conduit, and a powerhead. At least that portion of the signal transmission conduit that is adjacent to the powerhead is disposed within an interior of the support. This fourth aspect may include one or more of features discussed above in relation to one or more of the first through the third aspects, individually or in any combination, although any such additional feature(s) is not a requirement of the fourth aspect. For instance, the power injector of the fourth aspect may include one or more movable joints that incorporate a signal transmission connector having first and second components that move relative to each other in response to a movement between at least two different portions of the power injector.

### BRIEF DESCRIPTION OF THE FIGURES

FIGS. 1A and 1B are perspective views of one embodiment of a stand-mounted, dual-head power injector.

FIG. 2 is another embodiment of a stand-mounted, dual-head power injector.

FIG. 3 is a schematic of one of the rams and corresponding syringe that may be used by the dual-head power injector of FIG. 2.

FIG. 4 is a schematic of one embodiment of a pivot between a support and a powerhead, and that incorporates a signal transmission connector that is part of a signal transmission link associated with the powerhead.

FIG. 5 is one embodiment of a wall or ceiling-mounted power injector that utilizes a movable joint that incorporates a signal transmission connector that is part of a signal transmission link associated with the powerhead.

FIG. 6 is a plan view of another embodiment of a ceiling-mounted power injector and that may utilize at least one movable joint that incorporates a signal transmission connector that is part of a signal transmission link associated with the powerhead.

### DETAILED DESCRIPTION

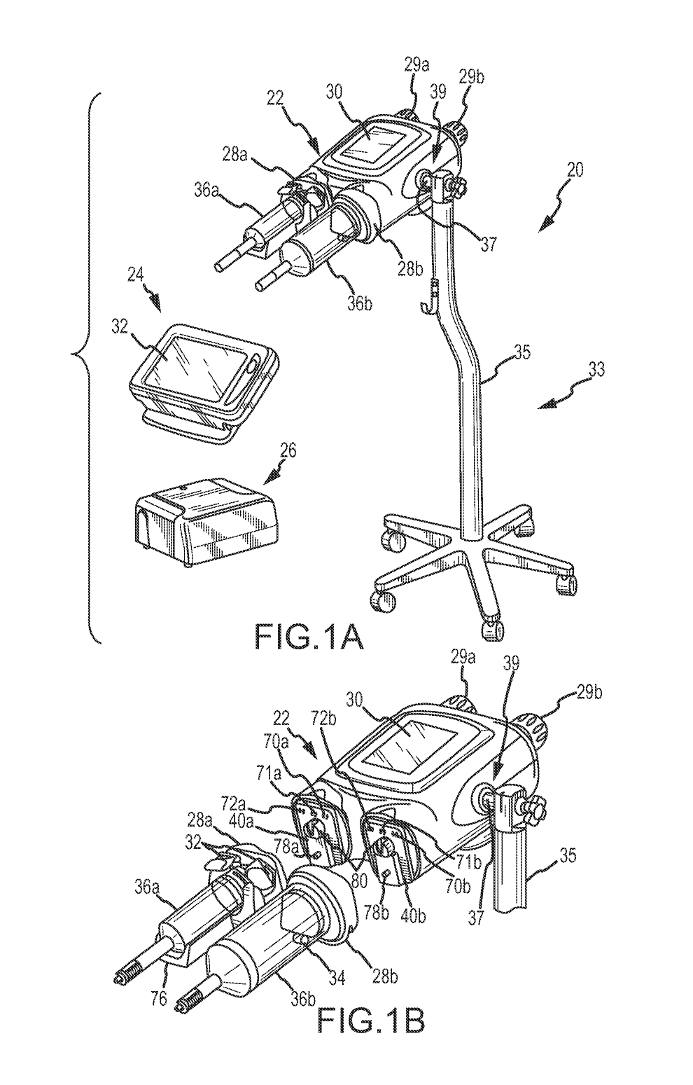

Referring to FIG. 1A, an injector 20 includes various functional components, such as a powerhead 22, a console 24 and powerpack 26. Syringes 36a and 36b are mounted to the injector 20 in faceplates 28a and 28b of the powerhead 22, and the various injector controls may be used to fill one or both of the syringes 36a, 36b with, e.g., contrast media for a CT, Angiographic or other procedure, which media is then injected into a subject under investigation under operator or pre-programmed control. Although the powerhead 22 is in the form of a dual head injector, it could also be in the form of a single head injector as well (e.g., for accommodating a single syringe).

The injector powerhead 22 includes hand-operated knobs 29a and 29b for use in controlling the movement of the internal drive motors engaged to syringes 36a and 36b, and a display 30 for indicating to the operator the current status and operating parameters of the injector 20. The console 24 includes a touch screen display 32 which may be used by the operator to remotely control operation of the injector 20, and may also be used to specify and store programs for automatic injection by the injector 20, which can later be automatically executed by the injector 20 upon initiation by the operator.

Powerhead 22 and console 24 connect through cabling (not shown) to the powerpack 26. Powerpack 26 includes a power supply for the injector 20, interface circuitry for communicating between the console 24 and powerhead 22, and further circuitry permitting connection of the injector 20 to remote units such as remote consoles, remote hand or foot control switches, or other original equipment manufacturer (OEM) remote control connections allowing, for example, the operation of injector 20 to be synchronized with the x-ray exposure of an imaging system.

Powerhead 22 is mounted to a wheeled stand 33, which includes a portable base, a column 35 that extends at least generally upwardly from this base, and a support arm 37 that extends from the column 35 and that supports the powerhead 22 for easy positioning of the powerhead 22 in the vicinity of the examination subject. Console 24 and powerpack 26 may be placed on a table or mounted on an electronics rack in an examination room. Other installations are also contemplated however; for example, powerhead 22 may be supported by a ceiling, floor or wall mounted support arm.

Referring now to FIG. 1B, further details of the powerhead 22 can be seen. In FIG. 1B, faceplates 28a and 28b have been removed, illustrating details of the mountings 40a and 40b for the faceplates 28a, 28b. Two different faceplates 28a, 28b are shown in FIG. 1B. Faceplate 28a is designed for mounting a 125 ml syringe 36a, and uses mechanical structures similar to those disclosed in U.S. Pat. No. 6,929,619. These structures include movable arms 32, which rotate into and out of engagement with the cylindrical body of the syringe 36a to engage the syringe 36a. The syringe 36a is installed perpendicular to its long axis to engage a button on the rearward face of the syringe 36a with a jaw 80 on the end of the drive ram, as shown in the above-referenced U.S. Pat. No. 6,929,619. Faceplate 28a includes a cradle housing 76 within which the syringe 36a is installed. Cradle housing 76 provides mechanical support for syringe 36a, may house various sensors, or both.

Faceplate 28b is designed for mounting a 200 ml front-loading syringe 36b, and uses mechanical structures similar to those illustrated in U.S. Pat. No. 5,300,031. These structures include a rotating cam, rotatable via an externally-extending arm 34 to cause translation of the faceplate 28b relative to the mounting 40b. The rotating cam further includes engaging keys that mate to slots on a rearward end of syringe 36b so that rotation of the syringe 36b is linked to rotation of the cam and translation of the faceplate 28b. The translation of faceplate 28b relative to mounting 40b causes a button on a rearward face of the plunger in syringe 36b to translate into and out of engagement with a jaw 80 on the end of the drive ram, as shown in U.S. Pat. No. 5,300,031.

The circuitry internal to powerhead 22 includes, in the area of mountings 40a and 40b, Hall effect sensors for detecting magnets in the corresponding faceplate 28a, 28b. Specifically, there are Hall effect sensors on the circuit board internal to injector powerhead 22 at each of positions 70a/70b, 71a/71b and 72a/72b. These sensors detect the presence or absence of a magnet in the corresponding faceplate 28a, 28b adjacent to positions 70a/70b, 71a/71b and 72a/72b. The sensors at positions 71a/71b and 72a/72b are used to identify the corresponding faceplate 28a, 28b mounted to powerhead 22, that is, the presence or absence of a magnet in a faceplate 28a, 28b corresponding to these locations identifies the faceplate type and thus the syringe size being used on that side of the injector 20. The sensors at positions 70a/70b have an alternative purpose of identifying whether the corresponding faceplate 28a, 28b is closed or open. Mountings 40a and 40b include, at locations 70a and 70b, magnetic conductors for carrying magnetic flux to a sensor on an internal circuit board.

The faceplates 28a and 28b each include a magnet in the movable mounting structures, that is, faceplate 28a includes a magnet within one of the movable arms 32, and faceplate 28b includes a magnet within the internal rotating cam coupled to arm 34. These magnets are positioned so that when the corresponding syringe 36a, 36b and faceplate 28a, 28b are engaged for injection, the magnet aligns with the magnetic conductor at location 70a/70b, triggering the sensor internal to powerhead 22. Because the movable structures in the faceplates 28a, 28b are not as close to mountings 40a and 40b as magnets at locations 71a/71b and 72a/72b, and for this reason a magnetic conductor is included at locations 70a/70b to ensure flux is channeled to the sensors internal to powerhead 22.

Mountings 40a and 40b further include a magnetic conductor 78a and 78b, useable to identify whether the corresponding faceplate 28a, 28b has been connected to the A or B side of the injector powerhead 22. The magnetic conductors 78a and 78b carry flux from magnets internal to the corresponding faceplate 28a, 28b. These magnets have opposite polarities and/or only one conductor 78a/78b contains a magnet, so that the positioning of a faceplate on the A or B side of the injector 20 may be distinguished by a sensor in faceplates with appropriate sensing electronics, such as the 125 ml faceplate 28a.

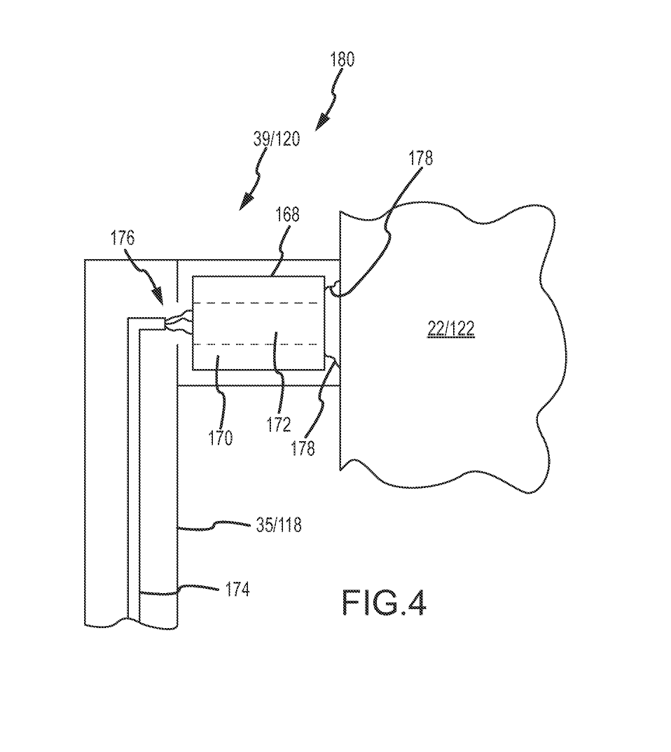

The position of the powerhead 22 relative to the stand 33 may be adjusted by a pivoting of the powerhead 22 about an axis coinciding with the long axis of the support arm 37. The interface between the support arm 37 and the powerhead 22 is in the form of a movable joint or pivot 39 that structurally supports the powerhead 22 on the stand 35. This pivot 39 may incorporate a signal transmission connector (not shown in FIGS. 1A, 1B, but discussed below in relation to FIG. 4) that is part of a signal transmission link to the powerhead 22. This signal transmission link may also include one or more cables for providing power to the powerhead 22, for transmitting data signals to the powerhead 22, for transmitting data signals from the powerhead 22, or any combination thereof. As will be discussed in more detail below, this signal transmission connector includes first and second components that move relative to each other in response to a relative movement between two different portions of the power injector 20.

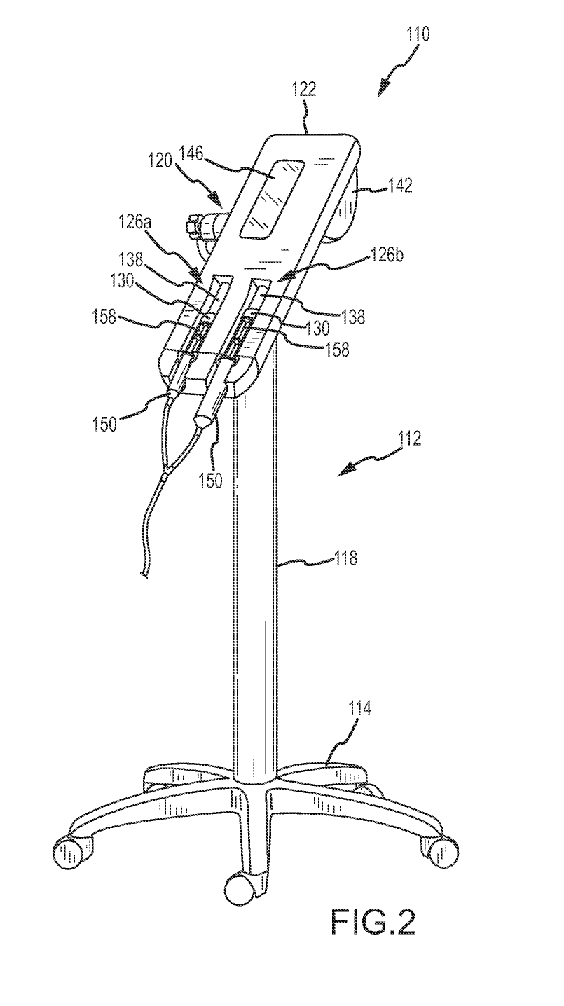

FIG. 2 is a perspective view of one embodiment of a power injector 110 that includes a support or stand 112, along with a powerhead 122 that is movably interconnected with the support 112 (e.g., pivotally, for instance to accommodate the powerhead 122 being in one position to draw or otherwise load a fluid into one or more syringes 150, and to further accommodate the powerhead 122 being in another position for an injection procedure). The support 112 may be of any appropriate size, shape, configuration, and/or type. The support 112 of the illustrated embodiment is in the form of a movable or portable base 114 (e.g., having a plurality of casters, rollers, or the like for portability), along with a column 118 that extends at least generally upwardly from the base 114. It should be appreciated that the support 112 need not include transportability functionality in all instances. Other configurations may be appropriate for the support 112. For instance, the support 112 could be adapted so as to be mountable to an appropriate structure (e.g., a wall, ceiling, or floor), could be adapted so as to include one or more positional adjustability features, or both.

The powerhead 122 may include an appropriate display or user interface screen 146 to accommodate providing one or more operational inputs to the power injector 110, to display various information, or the like. One or more other data input devices of any appropriate type could be integrated with the powerhead 122 outside of the display 146 as well (e.g., a remote console). The powerhead 122 is of a dual-head configuration, and thereby incorporates a pair of what may be characterized as syringe drivers 126a, 126b. Further in this regard, the power injector 110 includes a syringe 150 for each of the syringe drivers 126a, 126b. Typically, these syringes 150 will be detachably interconnected with (e.g., mounted on) the powerhead 122 in any appropriate manner, although such need not always be the case. Each syringe 150 may be of any appropriate size, shape, configuration, and/or type. Although the syringes 150 discharge into common tubing in the illustrated embodiment, such need not always be the case. The power injector 110 may integrate the powerhead 122 and syringes 150 in any appropriate manner, including without limitation using pressure jackets or without using pressure jackets. The powerhead 122 could also be adapted to utilize any appropriate number of syringes 150, including without limitation a single syringe 150 (e.g., a single-head configuration).

Each syringe driver 126a, 126b includes a ram 130 that is threadably engaged with a corresponding drive screw 138. Rotation of a given drive screw 138 axially advances its corresponding ram 130 along its long axis in a direction that is dictated by the rotational direction of the drive screw 138. The drive screws 138 are rotated through an operative interconnection with a motor 142 of the power injector 110, where the motor 142 may be of any appropriate size, shape, configuration, and/or type (e.g., an electric motor, a hydraulic motor, pneumatic motor, a piezoelectric motor).

Axial movement of a given ram 130 in the direction of its corresponding syringe 150 provides for a fluid discharge from this syringe 150, while an axial movement of a given ram 130 away from its corresponding syringe 150 accommodates, for instance, loading or an introduction of an appropriate fluid into this syringe 150, a removal of the syringe 150, or both. The ram 130 may be coupled with a plunger that is at least partially disposed within the syringe 150, such that movement of the ram 130 away from its corresponding syringe 150 retracts its associated plunger. In the embodiment of FIG. 2, however, the end of the ram 130 merely “butts up” against its corresponding syringe plunger. Therefore, advancing a ram 130 toward its corresponding syringe 150 in the FIG. 2 configuration will cause the ram 130 to engage its corresponding plunger to advance the same for an injection. However, retracting the ram 130 will cause the same to disengage its corresponding plunger, for instance such that the corresponding syringe 150 may be removed from the powerhead 122.

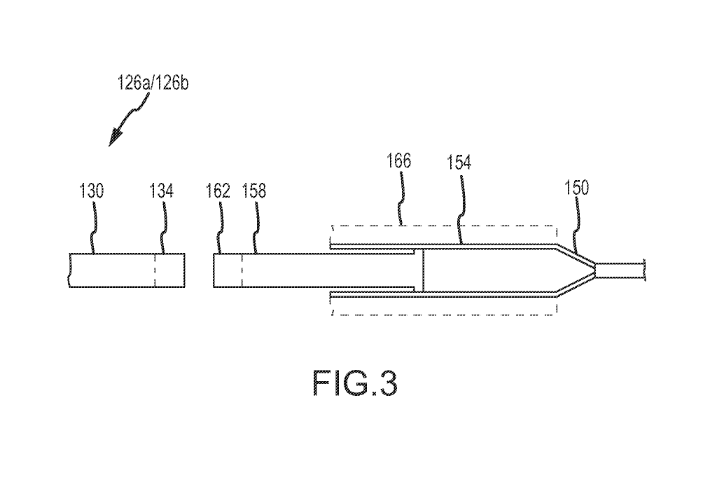

Each syringe 150 of the power injector 110 may be integrated with and supported by the powerhead 122 in any appropriate manner (e.g., detachably). Each syringe 150 may also be detachably coupled with its corresponding syringe driver 126a, 126b. This detachable coupling between a syringe 150 and its corresponding syringe driver 126a, 126b may be established in any appropriate manner, and is schematically presented in FIG. 3. Here, a ram 130 is schematically illustrated as having a coupler 134 on one of its ends, although such may not be required in all instances (e.g., for the FIG. 2 configuration). The syringe 150 is also schematically illustrated as having a syringe barrel 154 (which may be disposed in a pressure jacket 166 on the powerhead 122 as desired/required), along with an axially reciprocable syringe plunger 158 that extends within the syringe barrel 154 and that may include a coupler 162 on one of its ends (although such a coupler 162 may not be required in all instances (e.g., for the FIG. 2 configuration)). The ram coupler 134, along with the syringe coupler 162, each may be of any appropriate size, shape, configuration, and/or type. The detachable coupling between a ram 130 and its corresponding syringe plunger 158 may be established in any appropriate manner, as may be a decoupling of these two components. In one embodiment, an axial advancement of the ram 130 relative to the syringe plunger 158 may establish a coupling between the ram coupler 134 and the syringe coupler 162. Decoupling of the ram 130 from its corresponding syringe plunger 158 may be accomplished by moving (e.g., rotating and/or translating) the syringe 150 relative to the corresponding ram 130 in any appropriate manner. Generally, a movement between a syringe driver 126a, 126b and its corresponding syringe 150 in one or more dimensions may be used to establish a coupled or a decoupled condition between these components.

The power injector 110 may be used to discharge an appropriate fluid from each of the syringes 150 and in any appropriate manner (e.g., sequential discharges; simultaneous discharges). The power injector 110 may be used for any appropriate application, including without limitation for medical imaging applications. Representative medical imaging applications for the power injector 110 include without limitation computed tomography or CT imaging; magnetic resonance imaging or MRI; SPECT imaging; PET imaging; X-ray imaging; angiographic imaging; optical imaging; and ultrasound imaging. The power injector 110 could be used alone or in combination with one or more other components. The power injector 110 may be operatively interconnected with one or more components, for instance so that information may be conveyed between the power injector 110 and one or more other components (e.g., scan delay information, injection start signal, injection rate).

The position of the powerhead 122 relative to the support 112 may be adjusted for any appropriate purpose. One way to provide for this adjustment is illustrated in more detail in FIG. 4. FIG. 4 will be discussed in relation to the injector 110 of FIG. 2, although it is equally applicable to the injector 10 of FIGS. 1A and 1B. Therefore, reference numerals for both embodiments are presented in FIG. 4.

FIG. 4 illustrates a movable joint or pivot 120 that pivotally interconnects the column 118 of the support 112 with the powerhead 122, and this pivot 120 may be of any appropriate size, shape, and/or configuration. The pivot 120 structurally supports the powerhead 122 on the column 118. This pivot 120 incorporates an appropriate signal transmission connector 168. A number of characterizations may be made in relation to the signal transmission connector 168. One is that the signal transmission connector 168 may be characterized as being part of a signal transmission link that extends to the powerhead 122. Another is that the signal transmission connector 168 may be characterized as not being in the form of a load-bearing structure, such that the signal transmission connector 168 does not appreciably support the powerhead 122 on the column 118. Yet another is that the signal transmission connector 168 may be characterized as including first and second components that move relative to each other in response to a certain relative movement between two different portions of the power injector 110.

The above-noted signal transmission link to the powerhead 122 may provide power to the powerhead 122, may transmit data to the powerhead 122, may transmit data from the powerhead 122, or any combination thereof. This signal transmission link, in addition to the signal transmission connector 168, may also include one or more signal transmission conduits or cables 174. Representative signals that may be carried by any signal transmission conduit or cable 174 include power, data, or both. In the illustrated embodiment, the signal transmission connector 168 operatively interconnects a signal transmission conduit or cable 174 with the powerhead 122. In the illustrated embodiment, the signal transmission connector 168 is disposed within the interior of the pivot 120. Any appropriate number of wires or conductors 176 from the conduit 174 may be appropriately interconnected with the signal transmission connector 168. Any appropriate number of wires or conductors 178 may be interconnected with each of the signal transmission connector 168 and the powerhead 122.

The signal transmission connector 168 accommodates relative movement between the powerhead 122 and the column 118 of the support 112, preferably without causing any undesirable increase in stress in any of the wires 176, 178, and in any case while maintaining all desired signal transmission links between the signal transmission conduit 174 and the powerhead 122. For instance, the signal transmission connector 168 may be in the form of a slip ring or a slip ring module, such as the model AC6023 that is commercially available from Moog, Inc., specifically the Moog Components Group of Moog, Inc. having a business address of 1213 North Main Street, Blacksburg, Va. 24060-3127. Other slip ring module configurations may be appropriate.

In the illustrated embodiment, the signal transmission connector 168 includes an outer housing 170 (e.g., a first component) and an inner housing 172 (e.g., a second component) that are movable relative to each other (e.g., rotatable, such that the signal transmission connector 168 may be referred to as a rotary connector or as being of a rotary type), where there are one or more signal transmission links between the inner housing 172 and outer housing 170 (not shown), where the signal transmission conduit 174 is interconnected with one of the outer housing 170 or the inner housing 172, where the powerhead 122 is interconnected with the other of the outer housing 170 and the inner housing 172, and where there is an appropriate signal transmission link between each wire 176 (associated with the signal transmission conduit 174) and one or more of the wires 178 (associated with the powerhead 122). For instance, one or more wires 176 of the signal transmission conduit 174 may be appropriately interconnected with the inner housing 172, while one or more wires 178 may be appropriately interconnected with each of the outer housing 170 and the powerhead 122 (e.g., so that the wires 178 and outer housing 170 move along with, and in response to a movement of, the powerhead 122). Generally, pivoting the powerhead 122 relative to the column 118 will cause the outer housing 170 of the signal transmission connector 168 to rotate relative to the inner housing 172 of the connector 168. Stated another way, the signal transmission connector 168 maintains an electrical connection between the signal transmission conduit 174 and the powerhead 122, including during any relative movement between the powerhead 122 and the signal transmission conduit 174 and without generating any significant stress in the signal transmission conduit 174 during such movement. In one embodiment, the inner and outer housings 170, 172 of the signal transmission connector 168 move relative to each other about the same axis that the powerhead 122 moves. Generally, the signal transmission connector 168 allows one portion of the signal transmission link (e.g., wires 176) to remain stationary while another portion of the signal transmission link (e.g., wires 178) is allowed to move without experiencing any appreciable increase in mechanical stress (e.g., the wires 178 are not appreciably flexed or twisted by a movement of the powerhead 122—instead a portion of the signal transmission connector 168 moves).

Utilizing the signal transmission connector 168 may reduce stress in the electrical connection between the signal transmission conduit 174 and the powerhead 122 when changing the position of the powerhead 122. At least part of the signal transmission conduit 174 is disposed within an interior of the column 118 in the illustrated embodiment, although such may not be required in all instances. Disposing at least part of the signal transmission conduit 174 within the interior of the column 118 as it progresses toward the signal transmission connector 168 reduces the potential that an individual will grab onto the signal transmission conduit 174 and pull on the same in an attempt to move the power injector 110 from one location to another. Pulling on the signal transmission conduit 174 to move the power injector 110 may also stress the interconnection between the signal transmission conduit 174 and the powerhead 122. A powerhead zone 180 may be characterized as encompassing the powerhead 122 and at least that portion of the column 118 that is adjacent to the powerhead 122 (e.g., at least about 3 inches of an uppermost portion of the column 118). In one embodiment, all portions of the signal transmission link within the powerhead zone 180 are not exteriorly disposed, but are interiorly positioned. Stated another way, none of the signal transmission conduit 174 is exteriorly disposed throughout the powerhead zone 180 in one embodiment. Stated yet another way, at least that portion of the signal transmission conduit 174 that is disposed adjacent to the powerhead 122 may be located within the column 118.

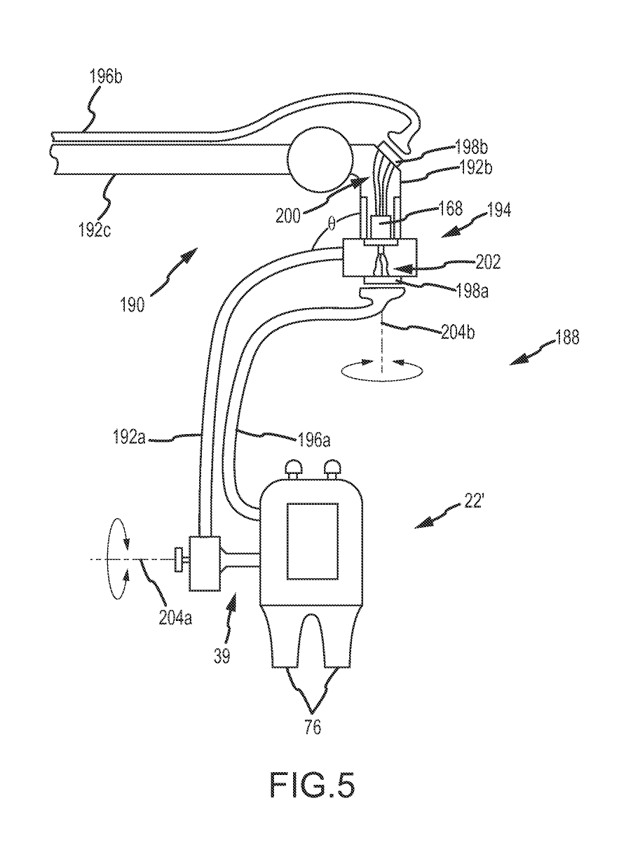

FIG. 5 illustrates an injector 188 that may be mounted to an appropriate structure (e.g., a ceiling, a wall, or a floor) in any appropriate manner. The injector 188 utilizes a variation of the powerhead 22 from the embodiment of FIGS. 1A-B. Therefore, the powerhead 22′ in FIG. 5 uses a “single prime” designation. Instead of using one cradle 76 as in the embodiment of FIGS. 1A-B, the powerhead 22′ in the FIG. 5 embodiment uses a pair of cradles 76—one for each syringe. Otherwise, the various features discussed above in relation to the powerhead 22 are equally applicable to the powerhead 22′.

The injector 188 includes a support 190 of any appropriate size, shape, configuration, and/or type that is mountable to an appropriate surface or structure in any appropriate manner. In the illustrated embodiment, the support 190 includes a support section 192a that is movably interconnected with the powerhead 22′ by a movable joint 39. The powerhead 22′ may be able to move at least generally about an axis 204a through a desired range of motion (e.g., motion of the powerhead 22′ may be constrained to at least substantially within a single dimension).

In the illustrated embodiment, the movable joint 39 does not incorporate a signal transmission connector 168, in contrast to the embodiment of FIGS. 1A-B. However, the injector 188 could be configured with the movable joint 39 having such an integrated signal transmission connector 168. A signal transmission conduit 196a used by the injector 188 of FIG. 5 could be disposed within the support section 192a as desired/required, and whether or not the movable joint 39 incorporates a signal transmission connector 168.

Another support section 192b of the support 190 is movably interconnected with the support section 192a by a movable joint 194 of any appropriate size, shape, configuration, and/or type. In the illustrated embodiment, the movable joint 194 allows the support section 192a to move relative to the support section 192b at least generally about an axis 204b that coincides with a length dimension of the support section 192b (e.g., motion of support section 192a relative to support section 192b may be constrained to at least substantially within a single dimension). The movable joint 194 may be configured such that any movement of the support section 192a relative to the support section 192b does not change the angle θ between the support sections 192a, 192b. A plug 198a of any appropriate type may be mounted on the movable joint 194 for interfacing/communicating with the signal transmission conduit 196a (e.g., a cable). Another plug 198b of any appropriate type may be mounted on the support section 192b for interfacing/communicating with another signal transmission conduit 196b (e.g., a cable). One or both of the signal transmission conduits 196a, 196b could be disposed inside a corresponding portion of the support 190.

A signal transmission connector 168 may be integrated with the movable joint 194, the support section 192b, or both. In the illustrated embodiment, the signal transmission connector 168 is disposed within the interior of each of the movable joint 194 and the support section 192b. One or more wires 200 extend from the plug 198b on the support section 192b to one portion of the signal transmission connector 168 (e.g., one of the outer housing 170 and inner housing 172 discussed above), while one or more wires 202 extend from another portion of the signal transmission connector 168 (e.g. the other of the outer housing 170 and inner housing 172) to the plug 198a on the movable joint 194. Generally and in accordance with the foregoing, the signal transmission connector 168 shown in FIG. 5 allows the wires 200 to move relative to the wires 202 in response to a relative movement between the support sections 192a and 192b without causing any undesirable increase in mechanical stress in any of the wires 200, 202. This movement of the wires 200 relative to the wires 202 furthermore is done while maintaining all desired signal transmission links between the plug 198a and plug 198b. Generally, the signal transmission connector 168 allows one portion of the signal transmission link (e.g., wires 200) to remain stationary while another portion of the signal transmission link (e.g., wires 202) is allowed to move without experiencing any appreciable increase in mechanical stress (e.g., the wires 202 are not appreciably flexed or twisted by a movement of the support section 192a—instead a portion of the signal transmission connector 168 moves).

Another support section 192c (e.g., a suspension arm) is appropriately interconnected with the support section 192b. The support sections 192c, 192b could be maintained in a fixed positional relationship relative to each other, or the support sections 192b, 192c could be movably interconnected in any appropriate manner (e.g., a movable joint that allows an angular position (e.g., measured in a vertical dimension) between the support sections 192b, 192c to be changed as desired/required). Although the support section 192c could be mounted to an appropriate structure, the support 190 may include one or more additional support sections (not shown) between the support section 192c and the structure to which the support 190 is mounted. One or more of these adjacent pairs of support sections could include a movable joint that incorporates a signal transmission connector 168 of the above-described type as desired/required.

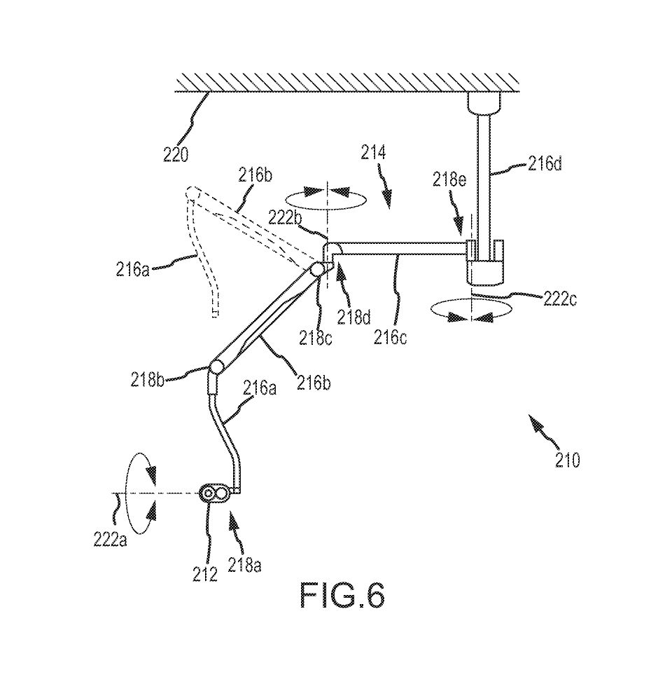

FIG. 6 illustrates an injector 210 that may be mounted in any appropriate manner to an appropriate structure 220, which in the illustrated embodiment is a ceiling. The injector 210 could be mounted to other structures, for instance a wall or a floor. In any case, the injector 210 includes a powerhead 212 of any appropriate size, shape, configuration, and/or type (e.g., a single-head configuration for accommodating a single syringe; a dual-head configuration for accommodating a pair of syringes). This powerhead 212 is movably interconnected with a support 214.

The support 214 may be of any appropriate size, shape, configuration, and/or type. In the illustrated embodiment, the support 214 includes a plurality of individual support sections 216a-d. The support section 216a is movably interconnected with the powerhead 212 by a movable joint 218a, for instance such that the powerhead 212 may be rotated or pivoted through a desired range of motion relative to the support section 216a about an axis 222a (e.g., motion of the powerhead 212 may be constrained to at least substantially within a single dimension). Movable joint 218b movably interconnects support sections 216a and 216b, for instance such that support sections 216a, 216b may be pivoted relative to each other as indicated by the dashed line shown in FIG. 6. Movable joint 218c movably interconnects support sections 216b and 216c, for instance such that support sections 216b, 216c may be pivoted relative to each other as indicated by the dashed line shown in FIG. 6. In the illustrated embodiment, the angular position of support section 216b in the vertical dimension may be varied, while the support section 216c may be maintained in a fixed position in the vertical dimension (e.g., a horizontal position in the illustrated embodiment).

Movable joint 218d also movably interconnects support sections 216b and 216c, for instance such that the support section 216b may be rotated or pivoted through a desired range of motion relative to the support section 216c about an axis 222b. Finally, movable joint 218e movably interconnects support sections 216c and 216d, for instance such that the support section 216c may be rotated or pivoted through a desired range of motion relative to the support section 216d about an axis 222c (e.g., motion of support section 216c may be constrained to at least substantially within a single dimension). A separate signal transmission connector 168 may be integrated with at least one of the movable joints 218a, 218d, and 218e. One embodiment has a separate signal transmission connector 168 being integrated with each of at least two of the movable joints 218a, 218d, and 218e. Another embodiment has a separate signal transmission connector 168 being integrated with each of the movable joints 218a, 218d, and 218e.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed is:

1. A power injector, comprising:
a support;
a cable comprising a first wire;
a powerhead, wherein said support provides mechanical support to said powerhead, and wherein said powerhead comprises a drive motor and an axially movable drive ram that is aligned with a location where a syringe may be installed on said powerhead; and
a pivot that extends between said support and said powerhead such that said powerhead is pivotable relative to said support, wherein said pivot comprises an electrical signal transmission connector which in turn comprises an outer housing and an inner housing that are rotatable relative to each other and that are electrically connected, wherein said first wire of said cable extends to and is electrically connected with one of said outer housing and said inner housing, and wherein the other of said outer housing and said inner housing is electrically connected with said powerhead by a second wire.

2. The power injector of claim 1, wherein said support comprises a portable base and a column that extends upwardly from said base.

3. The power injector of claim 1, wherein a first zone comprises said powerhead and at least a first section of said support that is adjacent to said powerhead, wherein none of said cable and none of said electrical signal transmission connector is exteriorly disposed throughout said first zone.

4. The power injector of claim 1, wherein at least part of said cable is disposed within said support.

5. The power injector of claim 1, wherein said inner housing and said outer housing each rotate about a first axis, and wherein said powerhead is also pivotable about said first axis.

6. The power injector of claim 1, wherein said electrical signal transmission connector is disposed within an interior of said pivot.

7. The power injector of claim 1, wherein said electrical signal transmission connector comprises a slip ring module.

8. The power injector of claim 1, wherein said electrical signal transmission connector comprises a rotary connector.

9. The power injector of claim 1, wherein said first wire remains stationary while said powerhead is pivoted relative to said support column and that moves said second wire relative to said first wire.
